(19) United States
(12) Patent Application Publication
(10) Pub. No.: US 2012/0280142 A1
(43) Pub. Date: Nov. 8, 2012

(54) WATER DISINFECTION BY ULTRAVIOLET RADIATION IN SOLAR ENERGY

(75) Inventor: Peter Johnstone, Victoria (AU)

(73) Assignee: FIRST GREEN PARK PTY LTD, Victoria (AU)

(21) Appl. No.: 13/513,144

(22) PCT Filed: Dec. 3, 2010
(86) PCT No.: PCT/AU10/01635

§ 371 Date: Jul. 24, 2012

(30) Foreign Application Priority Data
Dec. 3, 2009 (AU) 2009905902

(51) Int. Cl. A61L 2/10

(52) U.S. Cl. 250/435

(57) ABSTRACT

A solar energy water treatment device for minimizing bacteria and other pathogens in treatment water supplied to the device, the device having an inclined metal surface for receiving treatment water via a supply pipe at an upper end of the metal surface for flow downwardly thereon in a thin surface flow, a clear or translucent solar energy transfer panel being spaced above and adjacent to the metal surface whereby solar energy passes to the water on the metal surface, and water collection means at a lower end region of the metal surface after passage thereon, the treatment water flowing at a rate of between 0.1 and 2 litres/m²/min of the metal surface.

### PRIORITY CLAIM

The present application is a National Phase entry of PCT Application No. PCT/AU2010/001635, filed Dec. 3, 2010, which claims priority from Australian Patent Application No. 2009905902, filed Dec. 3, 2009, the disclosures of which are hereby incorporated by reference herein in their entirety.

### TECHNICAL FIELD

The present invention relates to the treatment of water by either filtering unwanted or undesirable solid particles or dissolved solids out of the water and/or to treat the water by ultraviolet radiation via solar energy to disinfect same or to destroy bacteria in the water.

### BACKGROUND ART

It is generally known to treat water chemically to destroy parasites and bacteria in the water. It is also known to use applied ultraviolet radiation to treat water to destroy parasites and bacteria in the water. It is also known to treat water contained in clear or translucent bottles with ultraviolet radiation contained in solar energy. Such solar energy works on pathogens in the water by the UV wavelength starting a photochemical reaction that targets DNA of bacteria in the water. Each of these known systems suffers from certain disadvantages. The addition of treatment chemicals can be costly, the chemicals may not always be available and moreover, the chemicals effectively remain in the water. Using an artificial source of ultraviolet radiation is not always possible and regardless there remains a need for an effective treatment regime. Utilizing solar radiation requires no fuel or chemicals, minimizes costs, but requires lengthy periods of solar energy exposure. Moreover, there is no effective mechanism for treating undesirable solids (dissolved or otherwise) in the water. It is often desirable to pre-treat water supplied to other devices such that dissolved and suspended solids will not harm such devices. Without limiting this aspect, such devices might include hot water heating devices for domestic and commercial applications. Further the treatment of bacteria and other pathogens in bottles by the use of solar energy does nothing for removal of solids (dissolved or otherwise) and is not a continuous or semi-continuous process.

### SUMMARY OF THE INVENTION

Embodiments of the present invention provide an improved method and apparatus for treating water to destroy or minimize bacteria and other pathogens in the water. Embodiments of the present invention provide a method and apparatus as aforesaid that can operate on a continuous or semi-continuous basis. Embodiments also provide a method and apparatus as aforesaid capable of removing dissolved or suspended solids in the water being treated.

Accordingly, embodiments of the present invention provide solar energy water treatment apparatus for minimizing bacteria and other pathogens in treatment water supplied to the apparatus, the apparatus comprising a metal surface receiving the treatment water via supply means at one end region for flow along the metal surface in a thin surface flow, a clear or translucent solar energy transfer panel spaced above and adjacent to the metal surface spanning the area of the metal surface on which said treatment water flows, a water collector for collecting the treatment water at an opposed end region of the metal surface after passage there along, the treatment water flowing at a flow rate of at least 0.1 litres/m²/min of the metal surface.

In an embodiment, the flow rate of the treatment water is between 0.1 and 2 litres/m²/min of the metal surface. Conveniently the flow rate of the treatment water is between 0.5 and 0.8 litres/m²/min of the metal surface. The metal surface, in use can have an angle of inclination of between 2° and 20° from the one end region to the opposed end region. Typically the angle of inclination is between 5° and 10°. It is generally desirable to maintain a slow flow rate to increase the temperature of the water to a level where bacteria or other pathogens will be destroyed by the time the water gets to the opposed end region ready for discharge.

In an embodiment, the metal surface may be an upwardly facing base surface of a metal foil tray, the tray conveniently being of an aluminium or aluminium alloy metal material. The metal surface may be contained within a treatment chamber bounded on an upper side by the solar energy transfer panel and on a lower side by a second panel spaced below but adjacent a lower face of the metal surface. The aforesaid panels may be formed by flexible sheets of plastic material contained at their edges by rigid frame members, the frame members also supporting the metal surface. Conveniently, the metal surface has a hydrophilic surface. Typically this may be achieved by the oxide layer formed on an aluminium or aluminium alloy surface.

In an embodiment, a porous removable filter layer may be positioned on the metal surface with an upper edge adjacent the treatment water supply to the metal surface, the filter layer having a lower edge spaced from the water collector. The removable filter layer may cover up to 100% but preferably 20-80% of the metal surface. If it is desired to minimize or destroy bacteria or other pathogens, the filter layer will end spaced a distance from the collector, so that a portion of the metal surface adjacent the collector will be free of the filter layer. If it is primarily desired to remove solids (dissolved or suspended) then the filter layer may extend to a position closely adjacent the collector. In an embodiment, the removable filter layer is formed from a biodegradable filter material. Such a biodegradable filter material may be a cellulose based material.

In yet another embodiment, the apparatus may include a sensor to sense a characteristic of the treatment water immediately upon discharge from the apparatus. Conveniently, the characteristic sensed may include any one or more of temperature, dissolved solids, suspended solids, bacteria or other pathogens.

Conveniently two or more such apparatus as previously described may be arranged in a series flow arrangement whereby treatment water collected from a first solar energy treatment apparatus may be delivered to the one end region of the metal surface of a second solar energy treatment apparatus. Conveniently bacteria and/or pathogens in the treatment water collected by the collector or the last said collector in a connected series of the treatment apparatus are destroyed.

### BRIEF DESCRIPTION OF THE DRAWINGS

Embodiments of the present invention will now be described with reference to the accompanying drawings, in which:

FIG. 1 is a partial cross-section of a lower end of a solar energy treatment apparatus according to an embodiment of the invention; and

FIG. 2 is a schematic illustration of several possible flow arrangements in an apparatus according to FIG. 1 or utilizing two or more such apparatus.

### DETAILED DESCRIPTION

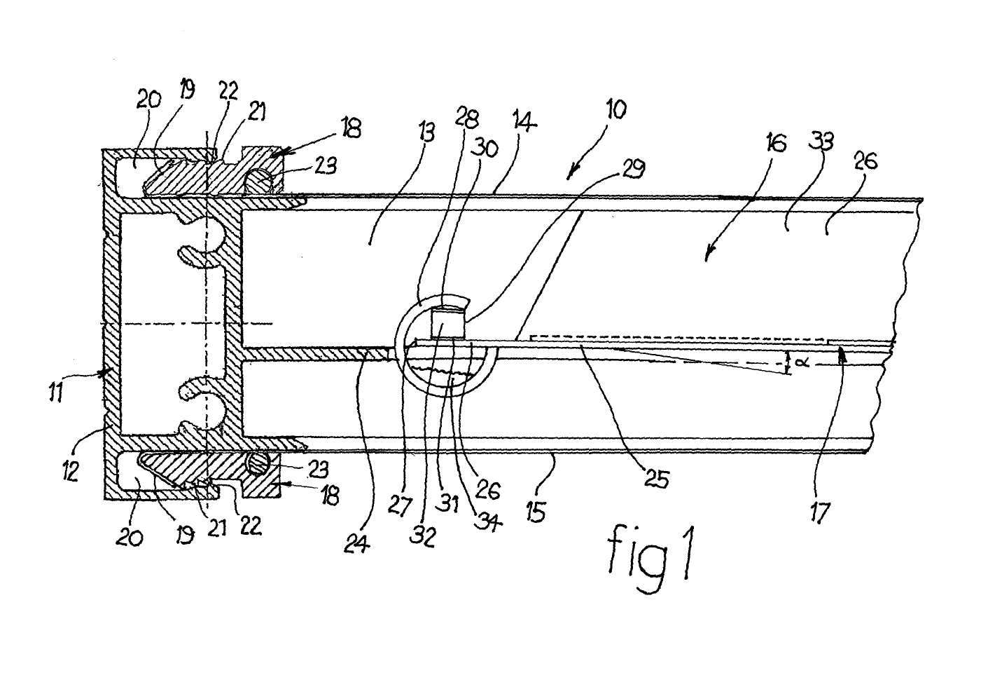

In accordance with an embodiment of the present invention shown in FIG. 1, the treatment apparatus 10 has a rigid perimeter frame 11 made from opposed end frame members 12 and opposed side frame members 13. The frame members 12, 13 are joined at corners to form a generally rectangular frame 11. FIG. 1 shows a longitudinal section through a lower end of the treatment apparatus 10 showing a lower end frame member 12 and a side frame member 13. The perimeter frame 11 carries an upper translucent or clear flexible plastic material panel 14 and a lower flexible plastic material panel 15 that may be clear translucent or opaque. The flexible panels 14, 15 together with the perimeter frame 11 define a treatment compartment 16 housing a treatment member 17 as further described below. The flexible panels 14, 15 are secured to the perimeter frame 11 by elongated clip elements 18 engaging preformed folds 19 in the edges of the panels 14, 15, with the clip elements 18 being engaged in recessed grooves 20 in the frame members 12, 13. The clip elements 18 may include a seal 23 such as an O-ring seal engaging an outer surface of the flexible panels 14, 15. Elongate projections 21 on the clip elements 18 engage with an inwardly directed projection 22 on an inner surface of the recessed grooves 20 whereby the position of the clip element 18 is adjustable in the recessed groove 20 to stretch the flexible panels 14, 15 so that in use, they are taut across the internal space defining the treatment compartment 16. The structure defined above may be as disclosed in Australian provisional patent application no. 2009905616 filed on 18 Nov. 2009 and in Australian provisional patent application no. 2010904294 filed on 23 Sep. 2010. The relevant subject matter from these applications are incorporated into the present specification by this reference thereto.

The treatment member 17 may be supported within the compartment 16 by flanges or other parts being supported on or by flanges 24 integrally formed with the frame members 12, 13. The treatment member 17 may conveniently have a base wall 25, side walls 33 and an upper end wall (not shown) generally in a tray like structure. The member 17 may also be supported by cross bars and/or longitudinal bars (not shown) engaging with the frame members 12, 13 and positioned below the base wall 25. Conveniently the treatment member 17 may be formed from a metal foil such as aluminium or aluminium alloy foil. Preferably the lower end 26 of the base wall 25 has no upstanding wall and terminates in an edge 27 located within a collection tube 28 mounted in the side frame members 13, one end of which, at least extends through the frame member 13 to discharge treatment water 34 from the apparatus 10. In this manner, all treatment water flowing downwardly on the upper surface of the base wall 25 is collected and discharged from the apparatus 10. Conveniently a member 29 holds the lower end 26 of the base wall 25 in the collection tube 28, the member 29 comprising upper and lower walls 30, 31 formed by spaced vertical walls 32.

In one arrangement, a delivery pipe 37, conduit or similar element extends to an upper end of the base wall 25 of the treatment member 17 to distribute treatment water across the upper end of the base wall 25 to allow it to flow downwardly on the wall 25 distributed as thinly and uniformly as possible across the upwardly facing surface of the base wall 25. The apparatus 10 may be inclined at an angle α so as to allow the water to flow downwardly on the surface while being impinged with solar energy passing through the upper wall 14 formed by the clear or translucent plastic panel or sheet 14. The angle α may be between 2° and 20°, preferably between 5° and 10°. This action treats the water with UV radiation within the solar energy to kill bacteria and pathogens in the water. Preferably the temperature of the water is increased by the applied solar energy to at least 30° C. and preferably in the range of 30° to 60° C. A temperature of 30° C. or higher may allow precipitation of dissolved salts such as calcium carbonate. The temperature of the water treated will depend on factors such as the atmospheric conditions, i.e. the level of applied solar energy, the inclination and the length of the treatment device. This may be varied by varying the inclination of the wall 25 which is preferably adjustable by adjusting the angle of inclination of the frame 11 of the apparatus 10. A treatment tray of the above described type may be of the order of 3 metres long by one metre wide giving a treatment area of about 3 m². The treatment water flow rate may be of the order of 0.1 to 2 litres/m²/minute depending on atmospheric temperature and applied solar energy. Conveniently the base wall of the tray may have a hydrophilic surface so as to allow the treatment water to spread evenly thereover. This may be achieved by an aluminium oxide layer if the tray is formed by an aluminium or aluminium alloy foil material. Other hydrophilic surface treatments may be applied to the upper surface of the wall 25.

If the treatment water has a reasonably high level of suspended solids, it is also desirable to provide a filter material layer 35 on at least the upper surfaces of the base wall 25 of the metal foil tray 17. The filter material 35 may be formed by a biodegradable material, for example a cellulose based material that is porous but will collect and retain the solids contained within the water. Eventually the filter material layer 35 may become clogged with the solids material and may then be removed, disposed of and replaced with a new filter material layer 35 In another possible embodiment the filter material layer 35 may be formed by a porous fabric material, preferably a non-woven fabric material that is hydrophilic to the treatment water. The fabric material may be a blend of 50% viscose and polyester or polypropylene (or alternatively 100% viscose). It may have a weight of 50 gm/m². In a further embodiment, the filter material layer might include a metal dispersed reasonably uniformly across the area of the material. In a particular arrangement, two layers of fabric material as aforesaid are utilized with metal dispersed between the two layers. Typically, the metal may be non corroding metal in the form of a mesh. The metal may be any one of aluminium, copper, stainless steel or alloys thereof.

Preferably collector 28 for the treated water 34 is positioned adjacent a lower end of the treatment tray. If a filter material layer is used it is preferred that its lower end is spaced above the collector. Conveniently the filter material layer may cover 20-80% of the base wall 25 of the treatment tray 17. If, however, the primary purpose of the apparatus 10 is to filter out suspended or dissolved solids in the treatment water, then the filter material layer might extend fully over the upper surface of the base wall 25.

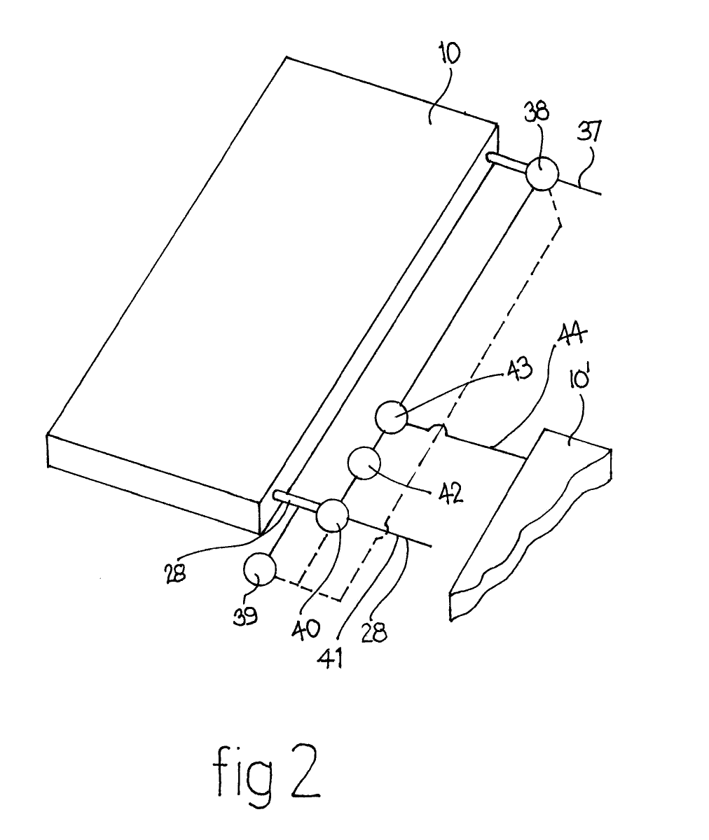

Reference may now be made to FIG. 2 of the annexed drawings where two similar treatment apparatus 10, 10′ are provided in a series flow arrangement. In this embodiment treatment water is supplied via a delivery pipe 37 via an inlet valve means 38 to the upper end of a treatment surface 25. The treatment water is collected at the lower end of the surface 25 in the discharge tube 28 and sensed by a sensor 39. The sensor 39 may sense a desired characteristic of the discharge water 34 which might be temperature which is an indicator of pathogen/bacteria destruction or may be some other characteristic such as solids content, pathogen/bacteria content or the like. If the sensed level of the characteristic sensed meets a required level, then a valve means 40 is positioned thereby to discharge the water via a supply line 41 to an end user. If the sensed level is unsatisfactory, the water may be recycled via the valve 40, a pump 42 to the inlet valve 38 to be recycled through the apparatus 10. In a further alternative arrangement, a valve 43 might direct the treatment water via an inlet passage 44 to the second treatment apparatus 10′. The treatment apparatus 10′ is conveniently constructed the same as the first apparatus 10.

It will of course be recognized that many variations are possible to the embodiments described above generally within the scope of the annexed claims.

1. Solar energy water treatment apparatus for minimizing bacteria and other pathogens in treatment water supplied to said apparatus, said apparatus comprising a metal surface receiving said treatment water via supply source 37 at one end region for flow along said metal surface in a thin surface flow, a clear or translucent solar energy transfer panel spaced above and adjacent to said metal surface spanning the area of said metal surface on which said treatment water flows, water collector 28 for collecting said treatment water at an opposed end region of said metal surface after passage there along, said treatment water flowing at a flow rate of at least 0.1 litre/m²/min of said metal surface.

2. Solar energy water treatment apparatus according to claim 1 wherein the flow rate of said treatment water is between 0.1 and 2 litres/m²/min of said metal surface.

3. Solar energy water treatment apparatus according to claim 2 wherein the flow rate of said treatment water is between 0.5 and 0.8 litres/m²/min of said metal surface.

4. A solar energy water treatment apparatus according to claim 1 wherein said metal surface has an angle of inclination of between 2° and 20° from said one end region to said opposed end region.

5. A solar energy water treatment apparatus according to claim 4 wherein said angle of inclination is between 5° and 10°.

6. Solar energy water treatment apparatus according to claim 1 wherein said metal surface is an upwardly facing base surface of a metal foil tray.

7. Solar energy water treatment apparatus according to claim 6 wherein said metal foil tray is an aluminium or aluminium alloy metal foil tray.

8. Solar energy water treatment apparatus according to any claim 1 wherein said metal surface is contained within a treatment chamber bounded on an upper side by said solar energy transfer panel and on a lower side by a second panel spaced below but adjacent a lower face of said metal surface.

9. Solar energy water treatment apparatus according to claim 8 wherein said panels are formed by flexible sheets of plastic material contained at their edges by rigid frame members, said frame members also supporting said metal surface.

10. Solar energy water treatment apparatus according to claim 1 wherein said metal surface is a hydrophilic surface.

11. Solar energy water treatment apparatus according to claim 1 wherein a porous removable filter layer is positioned on said metal surface with an upper edge adjacent treatment water supply means to said metal surface, said filter layer having a lower edge spaced from said water collection means.

12. Solar energy water treatment apparatus according to claim 11 wherein said removable filter layer covers 20 to 80% of said metal surface.

13. Solar energy water treatment apparatus according to claim 11 wherein said removable filter layer covers substantially all of said metal surface.

14. Solar energy water treatment apparatus according to claim 11 wherein said removable filter layer is formed from a biodegradable filter material.

15. Solar energy water treatment apparatus according to claim 14 wherein said biodegradable filter material is a cellulose based material.

16. Solar energy water treatment apparatus according to claim 1 including sensor 39 for sensing a characteristic of said treatment water immediately upon discharge from said apparatus.

17. Solar energy water treatment apparatus according to claim 16 wherein said characteristic includes any one or more of temperature, dissolved solids, suspended solids, bacteria or pathogens.

18. Solar energy treatment installation comprising at least first and second said solar energy water treatment apparatus according to claim 1, treatment water collected from said first solar energy treatment apparatus being delivered to the one end region of the metal surface of said second solar energy treatment apparatus.

19. Solar energy water treatment apparatus according to claim 1 wherein bacteria and/or pathogens in the treatment water collected by the collector or the last said collector in a connected series of said treatment apparatus are destroyed.
